(19) United States
(12) Patent Application Publication
(10) Pub. No.: US 2023/0338162 A1
(43) Pub. Date: Oct. 26, 2023

(54) MONOLITHIC CERVICAL OR LUMBAR IMPLANT BODY

(71) Applicant: NGMEDICAL GMBH, Nonnweiler (DE)

(72) Inventors: Hermann Backes, Namborn (DE); Nino Weiland, Nonnweiler (DE)

(21) Appl. No.: 17/925,707

(22) PCT Filed: May 26, 2021
(86) PCT No.: PCT/EP2021/064086

§ 371 Date: Nov. 16, 2022

(30) Foreign Application Priority Data
Jun. 4, 2020 (DE) 10 2020 114 862.3
Sep. 14, 2020 (DE) 10 2020 123 848.7

(51) Int. Cl. A61F 2/44; A61F 2/46

(52) U.S. Cl. CPC A61F 2/447; A61F 2/4611; A61F 2002/30593

(57) ABSTRACT

The invention relates to a monolithic cervical or lumbar implant body for fusing spinal column segments, comprising: an upper side and a lower side which are spaced apart from one another while maintaining an intermediate space, wherein the upper side and the lower side are connected by webs, wherein the upper side and the lower side, with the exception of shape-defining and structure-reinforcing edge regions, are designed as a honeycomb surface with openings and enclose a wedge-shaped angle; at least one completely or partially open side surface for introducing bone substitute material; and an additional side surface having a stabilisation region and a recess for temporarily receiving a surgical instrument in the stabilisation region. Starting from the surfaces of the shape-defining and structure-reinforcing edge regions, there are several pin-shaped projections oriented in the direction of adjacent spinal column segments. According to the invention, the recess is designed as a continuous or interrupted round thread.

The invention relates to a monolithic cervical or lumbar implant body for fusing spinal column segments, comprising an upper side and a lower side which are spaced apart from one another while maintaining an intermediate space, wherein the upper side and the lower side are connected by webs, wherein the upper side and the lower side, with the exception of space-defining and structure-reinforcing edge regions, are designed as a surface with openings and enclose an angle; at least one completely or partially open side surface for introducing bone material or bone substitute material, and an additional side surface having a stabilisation region and a recess for temporarily receiving a surgical instrument in the stabilisation region, according to the preamble of claim 1.

From the document US 2019/000636 A1, a vertebral implant having a unitary structure generated using a 3D printer is already known.

The document US 2008/249627 A1 relates to intermediate vertebral implants having a thread for being used in the human spinal column. The already known implants have functional threads serving the purpose of being fixed within the vertebra column.

From the generic document DE 10 2009 014 184 A1, an implant for fusing spinal column segments having a monolithic design is already known. At least parts of the surface of the implant have a structure-forming porosity. The volume of the implant has high density. Moreover, the volume comprises a number of direction-oriented and/or arbitrarily arranged passages pointing in different directions. The passages are surrounded, delimited or interrupted by stabilisation surfaces increasing the rigidity of the implant.

Preferably, the passages according to DE 10 2009 014 184 A1 are designed as a honeycomb structure. Such hexagonal passages represent a good ratio of the surface of the therewith created passage in the rigidity of the hollow space delimiting the structure.

Starting from the largest surface side of the monolithic implant as already known, the passages run in a perpendicular direction. In a preferred embodiment, the passages are interrupted by at least one free space in their direction-oriented course.

In a further embodiment, the passages run in one or several directions deviating from the perpendicular, whereby the caving behaviour is improved.

In a further development, the monolithic implant has a wedge-shaped profile which is slightly indicated, so as to facilitate the implantation process, on the one hand, and to conform to the shape of the spinal column in the anatomical respect, on the other.

The implant already known has a bore for temporarily receiving surgical instruments for facilitating the implant to be placed.

Moreover, at least one opening in the implant is present which serves the purpose of applying bone substitute material. The already known implant may be produced during a sintering process and/or by means of electron beam melting processes.

Additive processes for producing implants are basically further known. The so-called selective laser melting is used in this case.

In such a process, the material to be processed is applied in a thin layer in powder-form to a base plate.

This material is then locally remelted completely by means of laser beams and forms a solid material layer after rigidification. In this way, individual three-dimensional bodies also having undercuts and hollow spaces may be realized.

If in implants produced by means of additive processes threaded bores become necessary, so as for fix, for example, surgical instruments, it is hitherto required to design the threads either basically machined, i.e. cut, or to recut or regroove additively produced threads so as to secure corresponding precision. Thread-cutting or thread-recutting, however, requires the use of lubricants and a subsequent complex cleansing for removing the lubricants or for removing the cut chips so that additional working steps incur. Especially in case of miniaturized implant bodies, this is often combined with a considerable effort, difficulties, and costs.

It has been moreover shown that already known implant bodies having a honeycomb-structure according to the kind depicted initially, result in a subsidence, i.e. a caving of the implant into the spinal body due to a high surface pressure.

From the aforementioned, it is therefore a task of the invention to propose a further developed, monolithic cervical or lumbar implant body for fusing spinal column segments which avoids an undesired caving into adjacent spinal bodies or spinal column segments while using the honeycomb-structure which is per se advantageous, is nevertheless fixed securely, and moreover is constructionally implanted in such a manner that a production with purely additive processes is possible without any reworking.

A solution of the task of the invention is performed by an implant body having the feature combination according to claim 1, wherein the subclaims represent at least appropriate configurations and further developments.

A monolithic, cervical or lumbar implant body is consequently taken as a basis serving for fusing spinal column segments.

While maintaining an intermediate space, this implant body has upper and lower sides which are correspondingly spaced from one another.

The upper side and the lower side are connected by webs. With the exception of space-defining and structure-reinforcing edge regions, the upper and lower sides are designed as a surface, e.g. a honeycomb-surface, having passages, and are able to enclose a wedge-shaped angle.

At least one side surface is completely or partially open and serves for introducing bone substitute material or bone chips.

A further side surface has a stabilisation region. This stabilisation region is constituted as a correspondingly reinforced web connecting the upper and lower side of the implant body. The side surface may be bevelled at the transition to the lower side.

A recess is present in the stabilisation region. This recess serves for receiving a surgical instrument during the placement of the implant.

From the surfaces of the shape-defining and structure-reinforcing regions, several pin-shaped or mandrel-shaped projections having rounded tips are oriented in the direction of adjacent spinal column implants. In other words, these projections essentially extend perpendicularly from the corresponding surface of the structure-reinforcing edge region.

The recess for receiving a surgical instrument is formed according to the invention as a continuous or interrupted round thread. Here it is preferably a round thread of two radii merging tangentially into one another. Hereby, sharp edges, corners or overhangs are avoided due to the flank shape both on the implant and the associated instrument.

Due to the fact that there are no filigree flanks in a round thread and it does not have a large pitch, such a thread is very robust which represents a considerable advantage when placing the corresponding implant with the manipulation of the instruments.

Furthermore, the honeycomb wall thickness of the honeycomb structure is only a fraction of the honeycomb size. The honeycomb size here is defined as the distance between opposite honeycomb walls of a viewed honeycomb.

Preferably, a ratio between the honeycomb wall thickness and the honeycomb size is 1:10.

The honeycomb wall thickness may be designed to be differently thick targeted to the loads acting upon the implant. The honeycomb direction may be realized to be perpendicular or at a deviating angle. Likewise, some honeycomb walls may be omitted in a targeted manner so as to create a partial passage corresponding to the surface of two or more honeycombs.

In a further development of the invention, the upper side has a spherical, essentially convex surface shape. The lower side, on the contrary, is formed as a bevelled surface.

The angle of the bevelled surface in this case is in a range from essentially 0 to 15 degrees.

In a possible configuration of the implant body, the surface dimension of the upper side is smaller than that of the lower side.

The stabilisation region with the recess and the round thread is located essentially centrally in the further side surface.

In at least one of the webs connecting the upper side and the lower side, a recess or a setback is formed for guiding an instrument. A further, additional recess is arranged to be eccentrical and asymmetrical, and ensures together with a correspondingly designed instrument a positionally correct fixing of the implant on the instrument.

The mentioned protrusions preferably formed in a pin-shape or mandrel-shape are located in the corner regions of the implant body.

The protrusions have a rounding at their free ends. Thus, a wedge tip is not created but rather the shape of a mandrel or truncated cone having a rounded upper cone tip. Due to this formation of the protrusions, the implant is securely held in the corresponding adjacent spinal column body without creating a punctual wedge action which potentially damages the corresponding spine in a long-lasting manner.

At least one of the side surfaces, in particular that one which is opposite the side surface having the recess, is designed to be closed and does not form a product labelling surface. This labelling surface serves for receiving details, inter alia as to the producer, the charge (LOT) or further details.

According to the invention, the monolithic cervical implant body including the round thread is produced additively solely by selective laser melting without any subsequent machining methods or method steps. Reworks, in particular even in the thread region, are not required.

Hereinafter, the invention will be explained in more detail by means of exemplary embodiments and using Figures.

Shown are in:

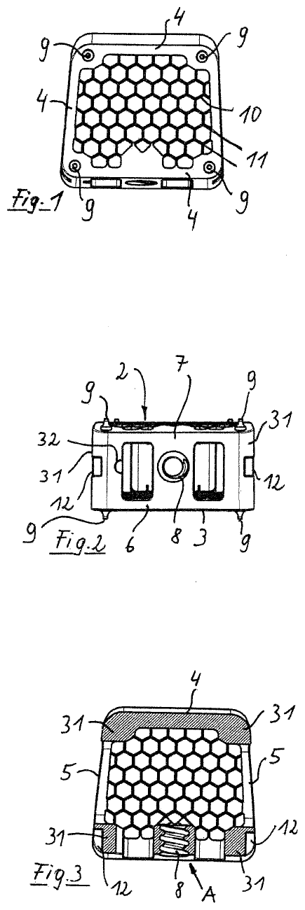

FIG. 1 a top view onto the upper side of an exemplary implant body;

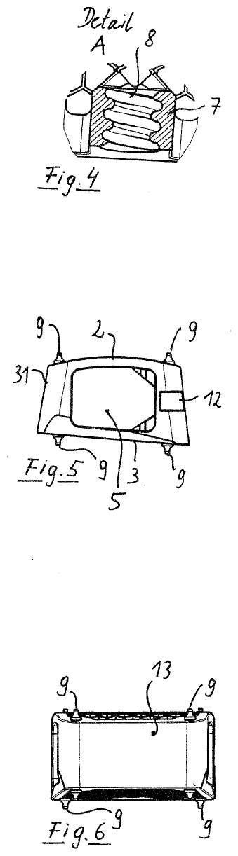

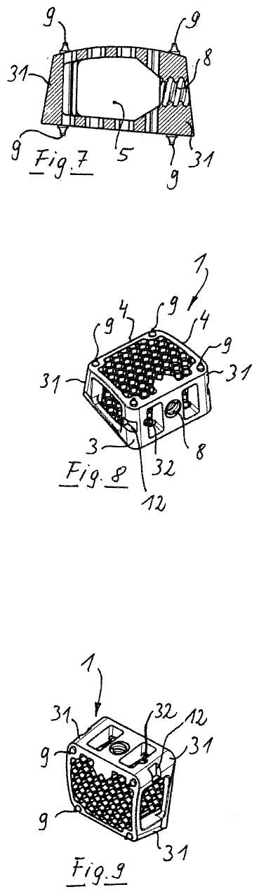

FIG. 2 a view of the further side surface having the stabilisation region and the thread recess;

FIG. 3 a sectional view along the lines A-A according to FIG. 2 having a recognizable round thread;

FIG. 4 a detailed view of the thread according to FIG. 3 (Detail A);

FIG. 5 a side view onto the implant body having an open surface for introducing bone substitute material in addition to recognizable protrusions at the lower side and upper side, as well as the spherical, convex surface shape of the upper side and the formation of the lower side as a wedge surface or bevelled surface;

FIG. 6 a representation of the closed implant backside surface with the possibility for product labelling being provided there;

FIG. 7 a sectional view along the line B-B according to FIG. 2, likewise having a recognizable round thread and protrusions;

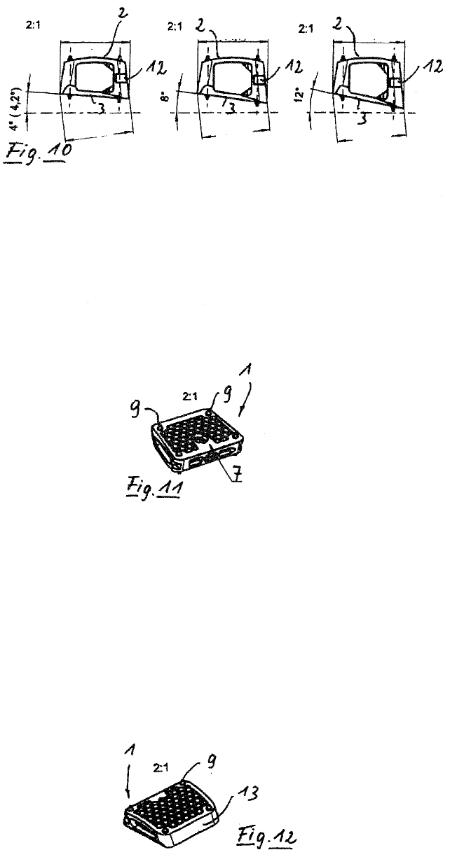

FIGS. 8 and 9 perspective representations of the implant body in different views:

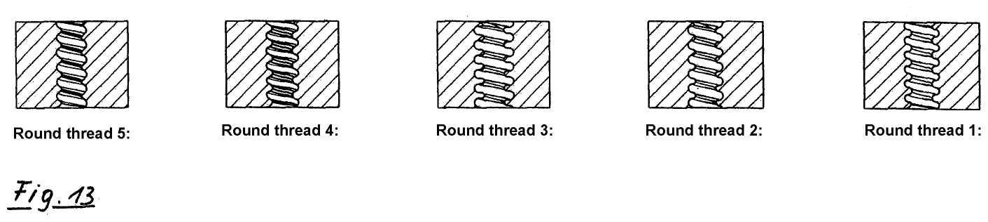

FIG. 10 exemplary representations of various angles of bevelled surfaces in the range from 4.2 to 12 degrees;

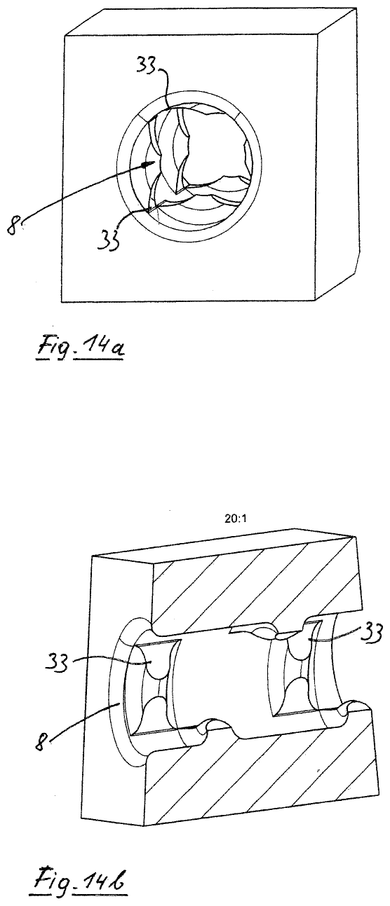

FIGS. 11 and 12 perspective representations in different views of a further embodiment of the implant body according to the invention having less height extension as compared to the exemplary embodiment according to FIGS. 1 to 9;

FIG. 13 examples of round thread configurations 1 to 5; and

FIGS. 14a, b representations of an exemplary interrupted round thread.

As far as dimensions are indicated in the figurative representations, these are to be understood as being merely exemplary and not limiting or restricting the idea of the invention. The same applies to indications as to the dimension in the figurative representations.

The monolithic implant body 1 has an upper side 2 and a lower side 3.

The upper side and lower side are connected by webs 31.

With the exception of edge regions 4 which are shape-defining and structure-reinforcing, the upper side 2 and the lower side 3 are designed as a honeycomb surface as in particular may be referred from FIGS. 1, 3, 8, 9 as well as 11 and 12.

The upper side 2, for example, has a spherical shape 2 and the lower side 3 has a bevelled surface, wherein the different angles of the bevelled surface as shown in FIG. 10 may be selected depending on the anatomic circumstances. For example, angles of 4.2 to 12 degrees are possible.

Opposite side surfaces 5 are designed to be mostly open so that substitute material or bone cement can be introduced into the interior free space of the implant body.

A further side surface 6 has a stabilisation region 7.

Within the stabilisation region 7, a recess is present designed as a round thread. See in this respect the representation according to FIG. 3 having the detail A according to FIG. 4.

Starting from the surfaces of the shape-defining and structure-reinforcing edge regions 4, there are several pin-shaped protrusions 9 oriented toward adjacent spinal column segments.

The honeycomb wall thickness 10 amounts only to a fraction of the honeycomb size 11 which is defined as the distance between opposite honeycomb walls.

For example, the honeycomb thickness preferably is 0.15 mm and the honeycomb size is 1.5 mm, so that the result is an exemplary ratio of 1:10.

It can be seen from the Figures that the surface dimension of the upper side 2 can be larger than the surface dimension of the lower side 3.

The stabilisation region 7 having the recess and round thread is essentially located centrally in the further side surface 6 as can be referred from FIGS. 1 to 3 as well as 8 and 9 but also 11.

In one of the webs 31 connecting the upper side 2 to the lower side 3, a recess or a setback 32 is designed for guiding instruments.

The protrusions 9 preferably are located in the corner regions of the respective implant body 1.

At least one of the side surfaces is configured to be closed and forms a product labelling surface 13. The closed side surface, i.e. the product labelling surface 13, preferably is realized to be opposite the stabilisation region 7 having the recess and round thread 8.

The implant body shown in the exemplary embodiments is manufactured including the round thread 8 additively by means of selective laser melting. Machining or other shape-changing methods for processing or re-working in particular of the round thread are not required.

On the basis of FIG. 13 respectively showing sectional views, one example of round thread configurations 1-5 will be explained.

The sectional view having the round thread 1 is a thread root radius which is equal to the radius at the flank tip. A direct transition between the thread root radius and the radius of the flank tip is given.

In the round thread 2, the thread root radius is larger than the radius at the flank tip. There is a thin flank. Furthermore, there is a direct transition between the thread root radius and the radius of the flank tip.

In the round thread 3, the thread root radius is larger than the radius at the flank tip. There is a thin flank. Furthermore, a perpendicular region between the thread root radius and the flank tip is given. The flank angle here amounts to approximately 0 degrees. The flank is perpendicular to the thread longitudinal axis.

In the round thread 4, the thread root radius is equal to the radius at the flank tip. The flank angle here deviates from 0 degrees.

In the round thread 5, the thread root radius is equal to the radius at the flank tip. The flank angle here deviates from 0 degrees.

FIG. 14a shows a perspective vies onto a recess having a round thread, wherein the round thread has interruptions 33. Thus, the round thread is interrupted or segmented for introducing the associated instrument in an easier manner.

FIG. 14b represents a longitudinal section of the view according to claim 14a having recognizable interruptions 33.

1. A monolithic cervical or lumbar implant body (1) for fusing spinal column segments, comprising an upper side (2) and a lower side (3) which are spaced apart from one another while maintaining an intermediate space, wherein the upper side (2) and the lower side (3) are connected by webs (31), furthermore the upper side (2) and the lower side (3), with the exception of space-defining and structure-reinforcing edge regions (4), are designed as a surface having openings and enclosing an angle, at least one completely or partially open side surface (5) for introducing bone substitute material, and a further side surface (6) having a stabilisation region (7) and a recess for temporarily receiving a surgical instrument in the stabilisation region,
characterized in that
the recess is formed as a continuous or interrupted round thread (8).

2. The monolithic cervical or lumbar implant body according to claim 1,
characterized in that
the surface is designed as a honeycomb surface, and the honeycomb wall thickness (10) as a fraction of the honeycomb size (11), defined as the distance between opposite honeycomb walls, preferably amounts to a ratio of essentially 1:10 between the honeycomb wall thickness (10) and the honeycomb size (11).

3. The monolithic cervical or lumbar implant body according to claim 1,
characterized in that,
starting from the surfaces of the shape-defining and structure-reinforcing edge regions (4) several pin-shaped or mandrel-shaped protrusions (9) extend in the direction of adjacent spinal column segments.

4. The monolithic cervical or lumbar implant body according to claim 1,
characterized in that
the upper side (2) has a spherical, convex surface shape, and the lower side (2) is designed as a bevelled surface.

5. The monolithic cervical or lumbar implant body according to claim 4,
characterized in that
the bevelled surface angle is in the range from essentially 1 to 15 degrees, preferably between 4 and 12 degrees.

6. The monolithic cervical or lumbar implant body according to claim 1,
characterized in that
the surface dimension of the upper side (2) is smaller than the surface dimension of the lower side (3).

7. The monolithic cervical or lumbar implant body according to claim 1,
characterized in that
the stabilisation region (7) having the recess and the round thread (8) is essentially located centrally in the further side surface (6).

8. The monolithic cervical or lumbar implant body according to claim 3,
characterized in that
in at least one of the webs (31) connecting the upper side and the lower side (2; 3), a recess or a setback (12) is formed for guiding an instrument.

9. The monolithic cervical or lumbar implant body according to claim 3,
characterized in that
the protrusions (9) each are located in the corner regions of the implant body (1).

10. The monolithic cervical or lumbar implant body according to claim 1,
characterized in that
at least one side surface is designed to be closed and forms a product labelling surface (13).

11. The monolithic cervical or lumbar implant body according to claim 8,
characterized in that
the closed side surface is opposite the stabilisation region (7) having the recess and the round thread (8).

12. The monolithic cervical or lumbar implant body according to claim 1,
characterized in that
it is additively manufactured including the round thread (8) without any machining methods by selective laser melting.
